(19) United States
(12) Patent Application Publication
(10) Pub. No.: US 2009/0270659 A1
(43) Pub. Date: Oct. 29, 2009

(54) METHOD FOR PRODUCING HYDROFLUOROCARBONS

(75) Inventors: Bertrand Collier, Saint-genis-laval (FR); Stephanie Amerio, Chaponost (FR); Michel Devic, Sainte Foy Les Lyon (FR)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: Arkema France, Colombes (FR)

(21) Appl. No.: 12/302,659

(22) PCT Filed: May 9, 2007
(86) PCT No.: PCT/FR2007/051235

§ 371 Date: Jun. 24, 2009

(30) Foreign Application Priority Data
May 30, 2006 (FR) 0604784
Jun. 21, 2006 (FR) 0605523

(51) Int. Cl. C07C 21/18; C07C 19/08; B01J 12/00

(52) U.S. Cl. 570/136; 570/134; 422/129

(57) ABSTRACT

The invention relates to a method for producing hydrofluorocarbons. The method comprises a step of reacting at least one hydro(fluoro)chlorocarbon or chlorocarbon with hydrofluoric acid in a gaseous phase in the presence of a catalyst, and a step of separating fluorination reaction products from the mixture. The method is characterized in that the gaseous flow from the reaction is compressed by means of a compressor before being subjected to the separation step. The invention also relates to a device for implementing the method.

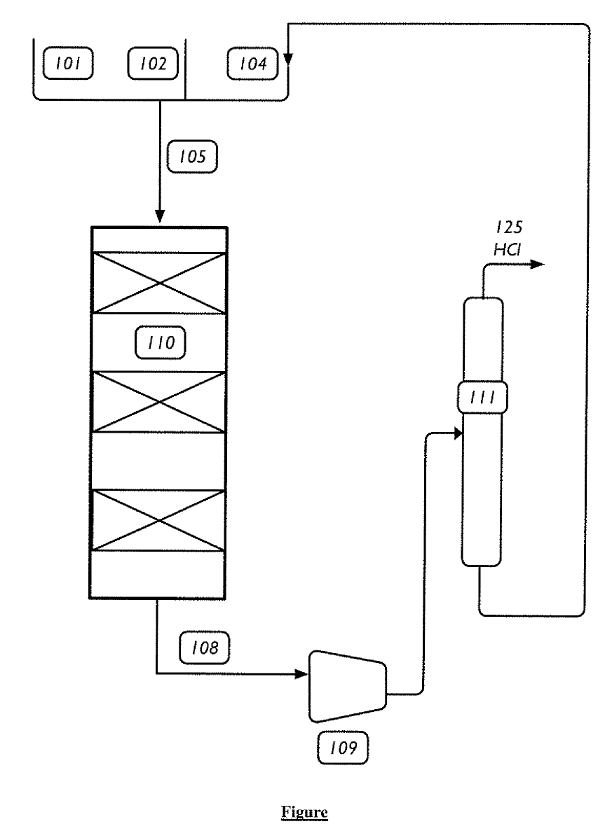

The present invention relates to a method for producing hydrofluorocarbons by reacting hydro(fluoro)chloro-carbons or chlorocarbons with hydrofluoric acid. It also relates to a device for implementing said method.

It is now established that, because of their considerable coefficient of action on ozone, chlorofluorocarbons will, in the long term, have to be replaced with refrigerant fluids that do not contain any chlorine. 1,1,1,2-tetrafluoroethane (134a), difluoromethane (32) and pentafluoroethane (125) are in particular used as chlorofluorocarbon substitutes.

Document EP 554165 relates to a continuous process for producing 1,1,1,2-tetrafluoroethane (134a) from 2-chloro-1,1,1-trifluoroethane and hydrofluoric acid. This document teaches the implementation of fluorination at a pressure of between 10 and 15 bar absolute in order to economically separate anhydrous HCl from the 134a.

Document EP 760808 describes a process for producing 1-chloro-1,2,2,2-tetrafluoroethane (124) as predominant product and also 1-chloro-1,1,2,2-tetrachloro-fluoroethane (124a) and pentafluoroethane (125) by reacting perchloroethylene (PER) with hydrofluoric acid (HF) in the gas phase in the presence of a catalyst in a reactor. The reaction product is subsequently subjected to distillation so as to give a distillate comprising hydrogen chloride (HCl), 124, 124a and 125 and a bottom fraction comprising PER, HF and organic intermediate compounds. Before it is recycled to the reactor, this fraction is subjected to a phase separation step in order to separate essentially HF from the mixture of PER and organic intermediates. This phase separation step is necessary for better control of the molar ratio of the reactants feeding the reactor.

Document EP 734366 describes a process for producing pentafluoroethane by reacting, in the first step, a perhaloethylene or pentahaloethane with HF in the gas phase in the presence of a catalyst. This document teaches the implementation of this step at a pressure that can range up to 30 bar absolute, in particular at a pressure of between 5 and 20 bar absolute, in order to facilitate the circulation of the gas stream in the plant.

Document EP 1110936 describes a method for preparing fluoroethane compounds by reacting at least one compound chosen from PER, dichlorotrifluoroethane (123) and 124 with HF in the presence of a chromium oxyfluoride catalyst having a fluorine content of at least 30% by weight. This document teaches carrying out the fluorination reaction at a pressure that will depend on the product-separation and purification conditions.

Similarly, document EP 1024124 teaches that, when the separation of 125 from the reaction products is carried out at a pressure above atmospheric pressure, the fluorination step is often carried out at high pressure.

Moreover, document EP 669303 describes a process for separating a gas mixture derived from a reaction for producing difluoromethane, by fluorination of methylene chloride with HF in the gas phase. This document teaches implementation by distillation and at high pressure, i.e. above 10 bar absolute, in order to efficiently separate the difluoromethane from the HF.

It is noted, in general, that the fluorination step in a process for producing hydrofluorocarbons of the prior art is often carried out at a pressure imposed by the operating conditions of the subsequent steps. It is also noted that the prior art recommends a high pressure in order to efficiently separate the fluorination reaction products.

The present invention provides a method for producing hydrofluorocarbons, comprising a step of fluorinating hydro(fluoro)chlorocarbons or chlorocarbons in the gas phase in the presence of a catalyst, and which does not have the constraints of the processes described in the prior art.

The method for producing hydrofluorocarbons comprises (i) a step during which at least one hydro(fluoro)-chlorocarbon or chlorocarbon react(s) with hydrofluoric acid in the gas phase in the presence of a catalyst, and (ii) a step of separating products derived from the fluorination step (i) from the mixture, characterized in that the gas stream from the fluorination step (i) is compressed by means of a compressor before being subjected to the separation step.

Preferably, the hydro(fluoro)chlorocarbon or chlorocarbon is chosen from dichloromethane, 2-chloro-1,1,1-trifluoroethane, 1,1,1,3,3-pentachloropropane, 1,1,1,3,3-pentachlorobutane, 1-chloro-1,2,2,2-tetra-fluoroethane, 1,1-dichloro-2,2,2-trifluoroethane and perchloroethylene. Dichloromethane, 2-chloro-1,1,1-trifluoroethane and perchloroethylene are advantageously chosen.

The fluorination step is advantageously carried out at an absolute pressure of between 1 and 5 bar. An absolute pressure between 1 and 3 bar is particularly preferred.

The temperature at which the hydro(fluoro)chlorocarbon or chlorocarbon reacts with hydrofluoric acid in the gas phase in the presence of a catalyst may be between 200 and 430° C., preferably between 250 and 350° C.

The HF/organic reactants molar ratio in the fluorination step may be between 5 and 60, preferably between 10 and 40, and advantageously between 15 and 25.

The fluorination step may be carried out in an isothermal or adiabatic reactor made from materials resistant to corrosion, for example Hastelloy and Inconel.

Any fluorination catalyst may be suitable for the method of the present invention. The catalyst used preferably comprises oxides, halides, oxyhalides or mineral salts of chromium, of aluminum, of cobalt, of manganese, of nickel, of iron or of zinc, it being possible for the catalyst to be supported.

A chromium oxide (Cr₂O₃)-based catalyst optionally including another metal in an oxidation state above zero and selected from Ni, Co, Mn and Zn is preferably used. Advantageously, this catalyst may be supported on alumina, fluorinated aluminum or aluminum oxyfluoride.

For this invention, mixed catalysts composed of nickel oxides, halides and/or oxyhalides and of chromium oxides, halides and/or oxyhalides, deposited on a support constituted of aluminum fluoride or of a mixture of aluminum fluoride and alumina, as described, for example, in patents FR 2 669 022 and EP-B-0 609 124, will be preferred.

When a mixed nickel/chromium catalyst is used, catalysts containing, by mass, from 0.5% to 20% of chromium and from 0.5% to 20% of nickel, and more particularly those containing from 2% to 10% by mass of each of the metals in a nickel/chromium atomic ratio of between 0.1 and 5, preferably in the region of 1, will be recommended.

The gas stream from the fluorination step is, in general, compressed to a pressure in the region of that of the separation step, preferably between 5 and 20 bar, advantageously between 10 and 15 bar. This makes it possible to carry out the separation step under favorable energy conditions and to recover most, preferably 99% by weight, of the hydrofluoric acid that has not reacted in the fluorination step.

Prior to the compression step, part or all of the gas stream from the fluorination step is preferably cooled so as to give a liquid phase and a gas phase. The gas phase is then subjected to the compression step and the liquid phase is pumped to the desired pressure. The compressed gas phase and also the liquid phase after pumping are subjected to the separation step.

The separation step preferably comprises a distillation step during which the hydrofluorocarbon compound and the hydrochloric acid are eliminated via the top of the column and unreacted hydrofluoric acid, the unreacted hydro(fluoro)chlorocarbon or chlorocarbon and also the intermediate compounds recovered at the bottom of the column can be recycled to the fluorination step.

The distillation step is preferably carried out at an absolute pressure of between 5 and 20 bar, advantageously between 10 and 15 bar.

The method of the present invention may be carried out continuously or batchwise, but it is preferred to operate continuously.

Although not necessary for the fluorination reaction, it may be judicious to introduce a small amount of oxygen or chlorine with the reactants. This amount may vary, according to the operating conditions, between 0.02 and 1 mol % relative to the reactants that go into the reactor. The introduction of the oxygen or of the chlorine may be carried out continuously or sequentially.

One embodiment of the invention is described with reference to the single FIGURE. A reactor (110), containing a supported chromium oxide-based catalyst, is fed by means of a gas stream (105) comprising, on the one hand, perchloroethylene (101) and hydrofluoric acid (102) and, on the other hand, unreacted HF, unreacted PER and intermediate compounds (123 and 124) that are recycled and derive from the stream (104). The gas stream (105) is preheated before introduction into the reactor maintained at a temperature of 350° C. The pressure in the reactor is approximately 3 bar absolute. The gas stream (108) leaving the reactor is first compressed by means of the compressor (109) at a pressure of approximately 15 bar absolute before being sent to the distillation column (111) so as to give, at the top, a fraction of light products comprising in particular pentafluoroethane and HCl, and at the bottom, a fraction of heavy products comprising HF, PER and intermediate compounds (predominantly 2,2-dichloro-1,1,1-trifluoroethane and 2-chloro-1,1,1,2-tetrafluoroethane). The fraction of heavy products leaves the distillation column via the bottom and is subsequently recycled to the reactor, whereas the fraction of light products is subjected to a distillation step in order to separate the HCl from the pentafluoroethane. The pentafluoroethane is subsequently purified.

A subject of the present invention is also a plant comprising in particular an evaporator (not represented), a reactor (110) containing the catalyst, reactant feeds, a compressor (109), a distillation column (111) for separating the HCl and the hydro-fluorocarbon at the top and recovering most of the unreacted hydrofluoric acid at the bottom of the column, and a distillation column (not represented) for separating the HCl from the hydrofluorocarbon. This plant can be used for the production of hydrofluorocarbons.

The present invention makes it possible to produce several different hydrofluorocarbons by means of the same plant. Moreover, the fact that the fluorination step is carried out under conditions independent of those of the separation step makes it possible to increase the lifetime of the catalyst.

1-7. (canceled)

8. A method for producing hydrofluorocarbons comprising the steps of:
(i) reacting at least one hydro(fluoro)chlorocarbon or chlorocarbon with hydrofluoric acid in the gas phase and in the presence of a catalyst to form a product gas stream;
(ii) compressing said product gas stream; and
(iii) separating at least one product from said product gas stream.

9. The method of claim 8, further comprising, prior to the compression step (ii), cooling part or all of said product gas stream to yield a gas phase and a liquid phase.

10. The method of claim 9, further comprising pumping said liquid phase before the separation step (iii).

11. The method of claim 8, wherein step (i) occurs at a pressure ranging from 1 bar absolute to 5 bar absolute.

12. The method of claim 11, wherein step (i) occurs at a pressure ranging from 1 bar absolute to 3 bar absolute.

13. The method of claim 8, further comprising compressing the product gas stream to a pressure ranging from 5 bar to 20 bar.

14. The method of claim 13, wherein the product gas stream is compressed to a pressure ranging from 10 bar to 15 bar.

15. The method of claim 8, wherein said at least one hydro(fluoro)chlorocarbon or chlorocarbon comprises dichloromethane, 2-chloro-1,1,1-trifluoroethane, 1,1,1,3,3-pentachloropropane, 1,1,1,3,3-pentachlorobutane, 1-chloro-1,2,2,2-tetrafluoroethane, 1,1-dichloro-2,2,2-trifluoroethane or perchloroethylene.

16. A plant for the production of hydrofluorocarbons comprising
an evaporator;
a reactor including a catalyst;
one or more reactant feeds;
a compressor;
a distillation column adapted to separate HCl and hydrofluorocarbon at the top of said distillation column and unreacted hydrofluoric acid at the bottom of said distillation column; and,
a distillation column adapted to separate HCl from hydrofluorocarbon.
